(19) United States
(12) Patent Application Publication
(10) Pub. No.: US 2014/0050710 A1
(43) Pub. Date: Feb. 20, 2014

(54) METHODS FOR PREPARING ACCESSORY CELLS AND USES THEREOF FOR PREPARING ACTIVATED NK CELLS

(71) Applicants: Martin Villalba Gonzalez, Montpellier (FR); Jean-Francois Rossi, Montpellier (FR); Zhao Yang Lu, Montpellier (FR)

(72) Inventors: Martin Villalba Gonzalez, Montpellier (FR); Jean-Francois Rossi, Montpellier (FR); Zhao Yang Lu, Montpellier (FR)

(73) Assignees: UNIVERSITE MONTPELLIER I, Montpellier Cedex 2 (FR); CENTRE NATIONAL DE LA RECHECHE SCIENTIFIQUE (CNRS), Paris (FR); INSTITUT NATIONAL DE LA SANTE ET DE LA RECHERCHE MEDICALE (INSERM), Paris (FR)

(21) Appl. No.: 14/114,241

(22) PCT Filed: Apr. 27, 2012
(86) PCT No.: PCT/EP12/57734

§ 371 Date: Oct. 28, 2013

(30) Foreign Application Priority Data
Apr. 28, 2011 (EP) 11305501.6

(51) Int. Cl. C12N 5/0783; A61K 35/14; C12N 5/071

(52) U.S. Cl. CPC C12N 5/0646; C12N 5/0602; A61K 35/17
USPC 424/93.71; 435/377; 435/373

(57) ABSTRACT

The present invention relates to a method for preparing accessory cells, said accessory cells may themselves be used for preparing activated NK cells that may be used in various therapeutic protocols (e.g. cancer treatment). More particularly, the present invention relates to a method for preparing an accessory cell comprising the steps consisting of i) providing a cell and ii) inhibiting in said cell the expression of a gene encoding for a Killer-Cell Immunoglobulin-like Receptor(s) (KIR) ligand.

### FIELD OF THE INVENTION

The present invention relates to a method for preparing accessory cells, said accessory cells may themselves be used for preparing activated NK cells for use in various therapeutic protocols (e.g. cancer treatment).

### BACKGROUND OF THE INVENTION

First lines of therapy for hematological malignancies generally included chemotherapy and/or chemo-immunotherapy with monoclonal antibodies particularly for B-cell malignancies. However, some patients experienced relapse or progression of the disease sometimes refractory to standard treatment. Some of them respond to a subsequent line of therapy but for a relatively short period of time.

Immune therapy including allogenic transplantation represents an alternative way for therapy in these patients after obtaining a second remission. Allogenic stem cell transplantation (SCT) from a sibling donor is only possible for 25% of the cases. Subsequent choices of haematopoietic stem cells include unrelated volunteers and unrelated umbilical cord blood (UCB) units. Treatment-related mortality represents the major problem and it could be reduced by progresses in supportive care and by using non myelo-ablative conditioning regimen (also designed reduced intensity conditioning regimen) in the transplant procedures, which allow its use in a larger number of patients. For example, in multiple myeloma, long-term follow-up studies demonstrated encouraging plateau in progression free survival up to 20% to 50% depending on selection criteria as disease status or performance status at allo-SCT. However, both graft versus host disease (GvHD) and (HvGD) severely decrease the success of such transplants.

High natural killer (NK) cell reconstitution is associated with reduced relapse and death without an increased incidence of GvHD after reduced intensity conditioning allogeneic hematopoietic stem cell transplantation. In contrast, there was no significant association between 60-day NK cell counts and clinical outcomes in patients receiving myeloablative conditioning.

NK cells are responsible of the graft versus leukemia (GvL) effect with minimal GvH and HvG effects, pointing attention to the development of immunotherapies involving NK cells. Data from several laboratories suggest that exploiting NK cell alloreactivity could have a large beneficial independently of NK cell source. Mismatched transplantation triggers alloreactivity mediated by NK cells, which is based upon ‘missing self recognition’. Donor-versus-recipient NK cell alloreactions are generated between individuals who are mismatched for HLA-C allele groups, the HLA-Bw4 group and/or HLA-A3/11. KIR ligand mismatching is a prerequisite for NK cell alloreactivity because in 20 donor—recipient pairs that were not KIR ligand mismatched in the graft-versus-host direction, no donor alloreactive NK clones were found.

In vitro studies on primary lympho-hematopoietic lineage tumor cells showed that living NK cells kill acute and chronic myeloid leukemia, as well as T-cell acute lymphoblastic leukemia (ALL), chronic lymphocytic leukemia, non-Hodgkin's lymphoma, and multiple myeloma. The only non-susceptible target was common, however, KIR-ligand incompatibility in the GvH direction improves outcomes after UCBT in patients. Importantly, clinical-grade production of NK cells has proven efficient, and NK cell-mediated therapy after hematopoietic cell transplantation seems safe. Therefore, the question is not if NK cells can eliminate leukemia cells in the clinic. Now the question is if we can generate industrial protocols to produce NK cells for clinical use and how using them. Regarding the later, an interesting idea is using non-myeloablative protocols that only reduced the bulk of leukemia cells followed by infusion of allogeneic NK cells. These NK cells will serve as mercenaries that will produce cytotoxicity only for a few weeks after infusion. These cells should eliminate residual leukemia cells in the patient preventing future relapse. This was the idea of a recent clinical trial that included infusion of haplo-identical KIR ligand mismatched and autologous stem cell transplantation. Moreover, other cancers, such as solid tumors (e.g. prostate cancer), could benefit for such approach.

However, some unresolved problems remain before the standard use of allogeneic NK cells in the clinic. Firstly, clinical studies show that engraftment of donor NK cells leads to patient remission, with a better outcome when a high number of cells is used. However, the number of NK cells obtained from a donor is limited and NK cell amplification under current conditions is poor. NK cells comprise 5 to 20% of human peripheral blood lymphocytes (PBMC). Considering that an average apheresis from a normal adult donor gives around 1×10¹° nucleated cells, a realistic number of NK cells obtained by donor is about 5×10⁸. Standard number of NK cells for infusion is 5×10⁷ NK cells/kg. Patients could receive more than one dose. This means that there is a need to amplify NK cells in vitro or in vivo to improve patient's prognostic. Secondly, in the current context, allogeneic NK cells improve the prognostic but do not induce remission or survival in 100% of patients. An interesting possibility to improve prognostic is injecting activated NK cells that possess a higher cytolytic activity than naive NK cells, but the % of activated NK cells in a healthy donor is low, around 5%. Several groups have begun clinical trials using in vitro expanded and/or activated NK cells, i.e: ClinicalTrials.gov Identifier: NCT00625729 (Masonic Cancer Center, University of Minnesota) and ClinicalTrials.gov Identifier: NCT00187096 (St. Jude Children's Research Hospital). In summary, protocols to amplify and/or activate NK cells in vitro will have obvious clinical applications.

The question is how expanding and/or activating NK cells. Several groups tried using different interleukins (IL) alone or in different combinations with very short clinical success. However, more efficient expansion is obtained with accessory cells of different origin. In fact comparisons of the gene expression profiles of the accessory-expanded NK cells and their IL-2-stimulated counterparts showed a different genetic remodelling with close to 1000 genes differently expressed. IL-2, or other cytokines, does not support clinical NK cell expansion in vitro, however, its co-injection with in vitro-expanded NK cells largely support both survival and activity of these cells.

One of the clinical trials in USA (NCT00187096) has generated K562 cells modified to express a membrane-bound form of IL-15 and the ligand of the costimulatory molecule 41BB with notable success. Seven-day coculture with these cells induced a 21-fold expansion of NK cells from peripheral blood. They expanded for up to 30 population doublings. Very interestingly for clinical purposes, these in vitro expanded activated-NK cells do not show cytotoxicity against non-transformed cells. Unfortunately, all these protocols equally expand all NK cells, independently of KIR expression and/or incompatibility against tumor cells.

The methods to activate and amplify NK cells described above induce proliferation and activation of the whole NK cell population. However, only a percentage of the donor NK cells will present mismatching. Moreover, only a percentage of these will effectively mediate allo-reactions. In a practical way, this is the population that should be amplified. We have developed and patented a protocol that allows selective amplification of the allo-reactive NK cell population. Here we propose to develop a second patent describing the accessory cells and the idea of NK cell banking.

Donor-versus-recipient NK cell reactivity is affected by a functional repertoire of NK cells that express inhibitory Killer-Cell Immunoglobulin-like Receptor(s) (KIR) for self class I ligand(s). KIRs sense missing expression of donor KIR ligand(s) in the recipient and mediate allo-reactions. The main inhibitory KIRs are KIR2DL1, KIR2DL2, KIR2DL3, KIR3DL1 and KIR3DL2. KIR2DL1 recognizes HLA-Cw4 and related, ‘group2’ alleles. KIR2DL2 and KIR2DL3 recognize HLA-Cw3 and related, ‘group1’ alleles. KIR3DL1 is the receptor for HLA-B allotypes with Bw4 motifs. Finally, KIR3DL2 is the receptor for HLA-A3/11. Inhibitory KIRs, CD94/NKG2, and HLA-class I genes determine individual NK cell repertoires during development. The HLA class I genotype selects a self tolerant repertoire by dictating which KIR and/or NKG2A receptor combinations are to be used as inhibitory receptors for self HLA class I. Consequently, functional NK cells in the mature repertoire express at least one inhibitory receptor for self HLA; coexpression of two or more receptors is less frequent. NK cells expressing receptors, which do not recognize self are retained in the repertoire in, however, an anergic (or ‘hypofunctional’) state.

Donor-versus-recipient NK cell alloreactions are generated between individuals who are mismatched for HLA-C allele groups, the HLA-Bw4 group and/or HLA-A3/11. Most donors have the potential to exert NK alloreactions as they possess a full complement of inhibitory KIR genes. HLA-C group1 receptor genes (KIR2DL2 and/or KIR2DL3) are present in 100% of individuals, the HLA-C group 2 receptor gene (KIR2DL1) in 97% and the HLA-Bw4 receptor gene (KIR3DL1) is found in 90% of individuals. The work from the Eurocord-Netcord and Acute Leukaemia Working Party of the EBMT gives an idea of the possibility of finding NK cell allogeneic donor between the european population. The probability of finding a NK cell allogeneic donor in an unrelated population is around 30%.

In a practical example, NK cells which express, as their only inhibitory receptor for self, a KIR for the HLA class group2 which is absent on allogeneic targets, sense the missing expression of the self class I KIR ligand and mediate alloreactions (‘missing self’ recognition). More specifically, 97% of individuals possess the KIR2DL1 receptor for HLA-C group2. If they possess HLA-C group2 allele(s) in their HLA type, they have HLA-C2-specific NK cells, which mediate alloreactions against cells from individuals who do not express HLA-C group2 alleles.

This means that if we want to specifically amplify and activate NK cells expressing one KIR, we can use target cells that do not express the specific KIR ligand. In a practical example if we want amplifying NK cells expressing only the KIR2DL1 receptor, we will generate target cells that lack HLA-C group2 alleles. Obviously, we could do the same for each individual KIR ligand, and therefore, produce NK cells expressing each specific KIR. This will be done for example by genetically modifying KIR ligand expression on the accessory cells by using small hairpin RNAs (shRNAs) against the specific HLA alleles: the MR ligands.

The protocol described above will expand and activate only NK cells with incompatibility against the selected tumor cells. To improve the efficiency of NK cell activation, we will also decrease expression of ERK5 in the accessory cells as described in the international patent application publication WO2009141729A2.

### SUMMARY OF THE INVENTION

The present invention relates to a method for preparing an accessory cell comprising the steps consisting of i) providing a cell and ii) inhibiting in said cell the expression of a gene encoding for a Killer-Cell Immunoglobulin-like Receptor(s) (MR) ligand.

### DETAILED DESCRIPTION OF THE INVENTION

The present invention relates to a method for preparing an accessory cell comprising the steps consisting of i) providing a cell and ii) inhibiting in said cell the expression of one gene encoding for a Killer-Cell Immunoglobulin-like Receptor(s) (MR) ligand.

According to the invention, any eukaryotic cell may be used. Preferably said cell is a mammalian cell. Typically said mammalian cells include but are not limited to cells from humans, dogs, cats, cattle, horses, sheep, pigs, goats, and rabbits. In a particular embodiment the cell is a human cell. In particular embodiment, the cell is a tumor cell obtainable from a patient. In another particular embodiment said cell is a cell line.

One essential feature of the invention is that the expression of one, and only one, type of MR ligand is inhibiting in said cell.

As used herein the term “MR” has its general meaning in the art and includes but is not limited to KIR2DL1, KIR2DL2, KIR2DL3, KIR3DL1 and KIR3DL2.

The KIR ligands are well known in the art. For example, KIR2DL1 recognizes HLA-Cw4 and related, ‘group2’ alleles. KIR2DL2 and KIR2DL3 recognize HLA-Cw3 and related, ‘group1’ alleles. KIR3DL1 is the receptor for HLA-B allotypes with Bw4 motifs. Finally, KIR3DL2 is the receptor for HLA-A3/11.

Any method well known in the art for inhibiting the expression or the activity of a gene may be used for inhibiting the expression of the gene encoding for a KIR ligand. By a way of example, said inhibition may be performed by using inhibitors of gene expression.

Inhibitors of gene expression for use in the present invention may be based on anti-sense oligonucleotide constructs. Anti-sense oligonucleotides, including anti-sense RNA molecules and anti-sense DNA molecules, would act to directly block the translation of KIR ligand mRNA by binding thereto and thus preventing protein translation or increasing mRNA degradation, thus decreasing the level of KIR ligand, and thus activity, in a cell. For example, antisense oligonucleotides of at least about 15 bases and complementary to unique regions of the mRNA transcript sequence encoding KIR ligand can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. KIR ligand gene expression can be reduced by contacting a subject or cell with a small double stranded RNA (dsRNA), or a vector or construct causing the production of a small double stranded RNA, such that KIR ligand gene expression is specifically inhibited (i.e. RNA interference or RNAi). Methods for selecting an appropriate dsRNA or dsRNA-encoding vector are well known in the art for genes whose sequence is known (e.g. see U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “ best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math. vol. 2, p: 482, 1981), by using the local homology algorithm developped by NEDDLEMAN and WUNSCH (J. Mol. Biol., vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p: 1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of KIR ligand mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Methods for delivering siRNAs, ribozymes and/or antisense oligonucleotides into cells are well known in the art and include but are not limited to transfection, electroporation, microinjection, lipofection, calcium phosphate mediated transfection, infection with a viral or bacteriophage vector containing the gene sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique may provide for the stable transfer of the gene to the cell, so that the gene is expressible by the cell, heritable and expressible by its cell progeny. Usually, the method of transfer includes the transfer of a selectable marker to the cells. The cells are then placed under selection to isolate those cells that have taken up and are expressing the transferred gene.

Typically, antisense oligonucleotides, siRNAs, shRNAs and ribozymes of the invention may be delivered in association with a vector. In its broadest sense, a “vector” is any vehicle capable of facilitating the transfer of the antisense oligonucleotide, siRNA, shRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In a preferred embodiment, the antisense oligonucleotide, siRNA, shRNA or ribozyme nucleic acid sequence is under the control of a heterologous regulatory region, e.g., a heterologous promoter.

In another embodiment, the method of the invention further comprises a step consisting of inhibiting or reducing the MHC-I expression in said accessory cell.

As a result said accessory cell will be presenting a negative MHC-I phenotype. According to the invention, accessory cells are considered as “presenting a negative MHC-I phenotype” for one KIR ligand when said accessory cells are capable of being lysed by syngenic NK cells (e.g. cells from the same individual or from a closely related immunologically compatible individual), including by non activated NK cells. The ability to be lysed by syngenic non activated NK cells is thus an indication that an accessory cell has an MHC-I negative phenotype.

The inhibition or reduction of the MHC-I expression is said accessory cell may be performed by any method well known in the art. For example said methods are exemplified in the international patent application publication WO2009141729A2. Typically, said inhibition or reduction of MHC-I expression is performed by using inhibitor of beta-2-microglobulin gene expression.

In another embodiment the method of the invention further comprises a step consisting of inhibiting the expression of ERK5 gene.

As a result said accessory cell will be presenting a negative ERK5 phenotype. The term “cell presenting a negative ERK5 phenotype” means a cell having a reduction of at least 10%, preferably 25% to 90%, for example 25% to 50% or 50% to 75% in the level of expression or the quantity of ERK5 protein present in the cell, in particular in the mitochondrial fraction, compared with its level of expression.

The inhibition or reduction of the ERK5 gene expression is said cell may be performed by any method well known in the art. For example said methods are exemplified in the international patent application publication WO2009141729A2. Typically, said inhibition or reduction of gene ERK5 expression is performed by using inhibitor of ERK5 gene expression.

In another embodiment, the method of the invention further comprises a step consisting of immortalizing said accessory cell.

As a result said accessory cell will constitute a cell line that proliferate indefinitely in culture. Methods for immortalizing cells are well known in the art. By a way of example, Epstein Barr virus (“EBV”) can immortalize human lymphocyte.

A further aspect of the invention relates to an accessory cell obtainable by the method of the present invention as above described.

A further aspect of the invention relates to a cell bank comprising at least one immortalized accessory cell as above described.

In a preferred embodiment said cell bank comprises a plurality of immortalized accessory cells according to the invention characterized in that said bank comprises i) at least one immortalized accessory cell wherein the expression of a gene encoding for KIR2DL1 ligand is inhibited, ii) at least one immortalized accessory cell wherein the expression of a gene encoding for KIR2DL2 ligand is inhibited, iii) at least one immortalized accessory cell wherein the expression of a gene encoding for KIR2DL3 ligand is inhibited, iv) at least one immortalized accessory cell wherein the expression of a gene encoding for KIR3DL1 ligand is inhibited and v) at least one immortalized accessory cell wherein the expression of a gene encoding for KIR3DL2 ligand is inhibited.

A further aspect of the invention relates to an in vitro or ex vivo method for preparing activated NK cells, comprising the steps consisting of (i) contacting living NK cells with at least one accessory cell of the invention under conditions and for a duration sufficient to induce activation of the NK cells; (ii) recovering said activated NK cells.

As used herein, the term “activated NK cells” means NK cells expressing several activation markers such as CD69, IFNγ, granzymes A and/or B, FasL and/or perforin, for example as evidenced using FACS. The levels of NK cell activation obtained using the methods of the present invention are significantly higher than levels which might be obtained when the activation is effected by contact of the NK cells with cytokines (for example IL-2, IL-12 or IL-15) or with alpha- or beta-interferons.

The living NK cells may be prepared from a donor, including umbilical cord blood units (UCB), by different techniques which are known by the skilled person. More particularly, these cells can be obtained by different isolation and enrichment methods using peripheral blood mononuclear cells (lymphoprep, leucapheresis, etc. . . . ). These cells can be prepared by Percoll density gradients, by negative depletion methods or by FACS sorting methods. These cells can also be isolated by column immunoadsorption using an avidine-biotin system or by immunoselection using microbeads grafted with antibodies. It is also possible to use combinations of these different techniques, optionally combined with plastic adherence methods. For example, the living NK cells can be prepared by providing blood mononuclear cells depleted of T cells from the donor, activating said cells with phytohemagglutinin (PHA) and culturing said cells with interleukin (IL)-2 and irradiated feeder cells.

The conditions of step i) for the NK cell activation may consist in culturing the living NK cells with different concentrations of accessory cells and in the presence of different concentrations of NK cell-activating cytokines such as IL-2, IL-12, IL-15, IL-18 and type I interferons. Only the NK cells missing the absence of their cognate receptor KIR ligand receptor in the accessory cells will be activated and, hence, will proliferate.

In one embodiment, the accessory cell is alive or has been previously irradiated.

The method as above described is particularly suitable for preparing activated NK cells with miss expression of one of the following KIRs: KIR2DL2 and KIR2DL3, KIR2DL1, KIR3DL1 and KIR3DL2. Consequently, the activated NK cells as above prepared will be alloreactive toward cells from others which lack the corresponding KIR ligand and, conversely, will be tolerant of cells from another individual who has the same KIR ligands.

A further aspect of the invention relates to a cell bank comprising a plurality of activated NK cells as prepared by the method of the present invention.

Accordingly, said activated NK cells may be then used in therapeutic protocols such as methods for treating cancers.

For example, the activated NK cells prepared according to the method are particularly suitable to enhance the efficacy and safety of allogeneic grafts. By a way of example, a method of transplanting allogeneic graft into a patient in need thereof may comprise the steps consisting of a) administering to said patient an effective amount of the activated NK cells as described above; and, b) transplanting the allogeneic graft into the recipient. This method of transplanting allogeneic graft, more particularly hematopoietic graft, can be applied for reducing the GVHD, for decreasing the intensity of the conditioning regimen, for treating a subject having hematologic disorder, more particularly leukemia, for treating or preventing an infection in a recipient of allogeneic graft, for enhancing immune reconstitution in an allogeneic graft recipient, for proceeding a hematopoietic graft with a greater T cell content, for increasing the engraftment, for reducing the graft rejection, for avoiding the tumor relapse and/or for conditioning a patient in need of a hematopoietic graft.

Hematologic disorder includes neoplastic proliferation of hematopoietic cells. Optionally, said hematologic disorder is selected from the group consisting of lymphoblastic leukemia, acute or chronic myelogenous leukemia, Hodgkin's lymphoma, Non-Hodgkin's lymphoma, myelodysplastic syndrome, multiple myeloma, and chronic lymphocytic leukemia. Preferably said hematologic disorder is a leukemia, more preferably myeloid leukemia. Hematologic disorder also includes non-malignant disorders such as an inherited erythrocyte abnormalities, an inherited immune system disorders or a hemoglobinopathy, e.g. sickle cell anemia, aplastic anemia or thalassemia.

For example, when activated NK cells shall be used in the treatment of hematological cancers, the physicians may perform HLA genotyping of patients. They could then order activated NK cells missing expression of the required KIRs (e.g. an AML patient has tumor cells that do not express ligands for KIR2DL1). NK cells expressing only one KIR (e.g. KIR2DL1) may then be provided and used in the therapeutic protocol.

Preferably, said allogeneic graft is a hematopoietic graft. Optionally, said hematopoietic graft is a bone marrow transplant. In a particularly interesting embodiment of the methods according to the present invention, said patient is treated for leukemia, more preferably myeloid leukemia, optionally an acute or chronic myeloid leukemia.

In one embodiment of the methods according to the present invention, the activated NK cells and allogeneic graft are administered simultaneously. In an alternative embodiment, the activated NK cells are administered prior to the allogeneic graft.

The efficient amount of activated NK cells administered to the recipient can be between about 0.05 10⁶ and about 100 10⁶ cells/kg of recipient's body weight. The efficient amount of hematopoietic cells administered to the recipient can be between about 0.2 10⁶ and about 10 10⁶ CD34+ cells/kg of recipient's body weight, In a preferred embodiment, the graft comprises a maximum of 1 10⁵ CD3+ cells/kg of recipient's body weight.

The activated NK cells and hematopoietic cells are typically administered to the recipient in a pharmaceutically acceptable carrier by intravenous infusion. Carriers for these cells can include but are not limited to solutions of phosphate buffered saline (PBS) containing a mixture of salts in physiologic concentrations.

The hematopoietic cells can be provided by bone marrow cells, mobilized peripheral blood cells or cord blood cells. The bone marrow cells can be obtained from the donor by standard done bone marrow aspiration techniques know in the art, for example by aspiration of marrow from the iliac crest. Peripheral blood stem cells are obtained after stimulation of the donor with a single or several doses of a suitable cytokine, such as granulocyte colony-stimulating factor (G-CSF), granulocyte/macrophage colong-stimulating factor (GM-CSF) and interleukin-3 (IL-3). In a preferred embodiment of the invention, the donor is stimulated with G-CSF. In order to harvest desirable amounts of stem cells from the peripheral blood cells, leukapheresis is performed by conventional techniques and the final product is tested for mononuclear cells. Optionally, the hematopoietic cells can be T-cell depleted. T-cell depletion of bone marrow or of peripheral blood cell may be carried out by any known technique, for example, by soybean agglutination and E-rosetting with sheep red blood cells as described.

According to the invention the host patient is conditioned prior to the transplantation of the allogeneic graft. Conditioning may be carried out under sublethal, lethal or supralethal conditions, for example by total body irradiation (TBI) and/or by treatment with myelo-reductive or myelo-ablative and immunosuppressive agents. According to standard protocols, a lethal dose of irradiation is within the range of 7-9.5 Gy TBI, a sublethal dose is within the range of 3-7 Gy TBI and a supralethal dose is within the range of 9.5-16 Gy TBI.

Any immunosuppressive agent used in transplantation to control the rejection, or a combination of such agents, can be used according to the invention, such as prednisone, methyl prednisolone, azathioprine, cyclophosphamide, cyclosporine, monoclonal antibodies against T-cells, e.g. OKT3, and antisera to human lymphocytes (antilymphocyte globulin—ALS) or to thymus cells (antithymocyte globulin—ATG). Examples of myelo-ablative agents that can be used according to the invention are busulphan, dimethyl myleran and thiotepa.

The advantage of the administration of active activated NK cells is the possibility to reduce the intensity of the conditioning regimen. For example, the conditioning regimen can be reduced to the intensity conditioning regimen adopted for matched human transplants. A reduced version of a highl-intensity regimen according to the present invention includes Fludarabine at the total dose of 200 mg/M2, Thiotepa 5 mg/Kg, and Melphalan 70 mg/M2, plus anti-T cell antibodies such as ATG, 20 mg/Kg. Optionally, the doses of Thiotepa and Melphalan can be increased by 50%. Indeed, such conditioning regimen is highly toxic and some patients are unable to withstand such toxicity. Therefore, the present invention makes possible the allogeneic graft for these patients.

The activated NK cells as prepared according to the invention may be used as first line of treatment in combination with standard total body irradiation (TBI) and/or by treatment with myelo-reductive or myelo-ablative and immunosuppressive agents; or used to control the residual disease after total body irradiation (TBI) and/or by treatment with myelo-reductive or myelo-ablative and immunosuppressive agents.

The activated NK cells as prepared according to the invention may also be used for the treatment of other NK-sensitive malignancies including pediatric cancers such as the sarcomas Ewing sarcoma and rhabdomyosarcoma (Cho . . . Campana clinical cancer Research 16:3901 (2010), neuroblastoma, malignant glioma and, possibly, prostate cancer (Cho, D. & Campana, D. Expansion and activation of natural killer cells for cancer immunotherapy. Korean J Lab Med 29, 89-96 (2009).

The activated NK cells as prepared according to the invention may also be used for the treatment of agressive cancer with no major drugs in order to improve the response rate and PFS. Typical said cancers include but are not limited to pancreatic cancer, lung cancer, breast and colon cancer.

The activated NK cells as prepared according to the invention may also useful for the treatment of infectious diseases or dysimmune diseases.

The invention will be further illustrated by the following figures and examples. However, these examples and figures should not be interpreted in any way as limiting the scope of the present invention.

### FIGURES

FIG. 1. Accessory cells are essential for productive NK cell amplification. UCB-derived lymphocytes (left panel) were incubated for ten days with IL-2 (1000 U/ml: middle panel) or with accessory cells (KT17 plus IL-2 (100 U/ml). In the upper panel, graphics show the proportion of lymphocytes (CD45+) that do not express CD3 (T cells) or CD56 (NK cells). NK cells (CD56+), which are in the left up panel, are readily amplified by accessory cells.

FIG. 2. Specific amplification of mismatched NK cells. We obtained lymphocytes from an UCB that express KIR2DL2/3 (CD158b: left panels) and KIR2DL1 (CD158a: right panels) with an haplotype HLA-Cg1, which is recognized by KIR2DL2/3, and HLA-Cg2 positive, which is recognized by KIR2DL1. This means that this UCB contains NK cells alloreactive against EBV cell lines missing HLA-Cg1 (PLH) or HLA-Cg2 (HOM-2). In this figure we incubated the lymphocytes with PLH cells to amplify NK cell expressing KIR2DL2/3 (CD158b). Ten days after incubation, we observed a small increase in this population compare to NK cells expressing KIR2DL1 (CD158a). This specific expansion was really apparent at day 20 where 30% of NK cells specifically expressed the selected KIR.

FIG. 3: Stimulation with feeder or accessory cells gives better NK amplification than high dose IL-2 stimulation. UCB mononuclear cells (after ficoll, 0.5-1.10e6 cell/ml) are stimulated with high dose interleukine-2 (IL-2, 1000 U/ml, dotted line) or feeder cells with medium dose IL-2 (100 U/ml, full line). Numbers represent the fold increase at day 10 compared to day 0. Figure shows NK (left), T lymphocytes (right, dark grey), NKT (right, medium grey) and B lymphocytes expansion (right, soft grey) for different experiments and with one UCB.

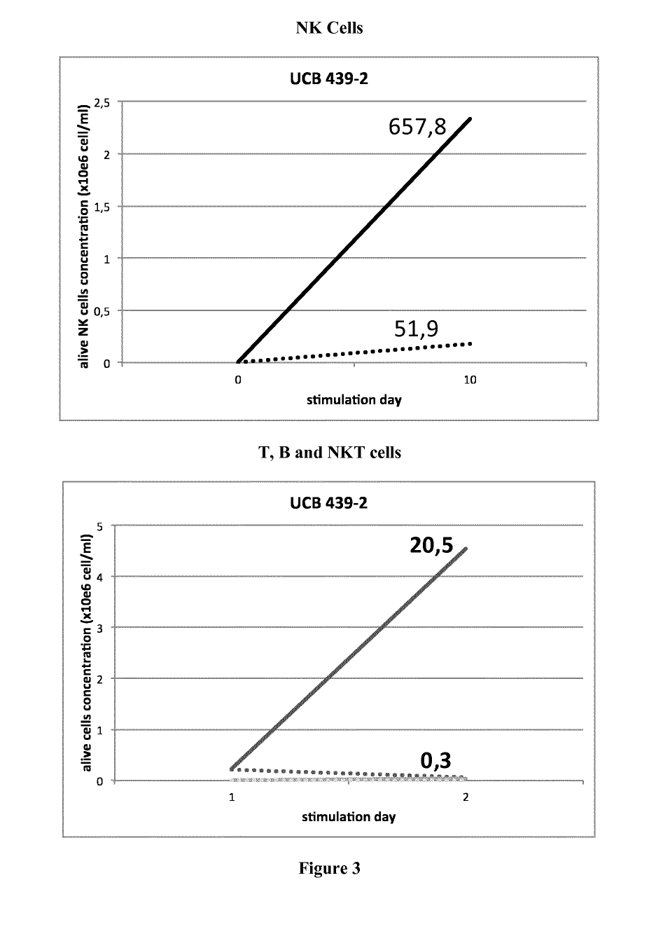

FIG. 4: Feeder cells give better NK amplification when used alive. The use of irradiated feeder cells during the first 3 days greatly improved NK cell expansion. UCB mononuclear cells (after ficoll, 0.5-1.10e6 cell/ml) are stimulated in different conditions. Comparison of IL-2 stimulation (1000 U/ml), direct incubation with alive feeder cells (IL-2 100 U/ml, UCB:F ratio 1:20), preactivation with irradiated feeder cells (IL-2 100 U/ml, UCB:F 1:1) and preactivation with IL-2 1000 U/ml.

### EXAMPLE 1

Most donors have the potential to exert NK cell alloreactions as they possess a full complement of inhibitory KIR. The genes for the HLA-C group1 receptor (KIR2DL2 and/or KIR2DL3) and the HLA-A3/11 receptor (KIR3DL2) are present in ˜100% of individuals; the genes for the HLA-C group2 receptor (KIR2DL1) and the HLA-Bw4 receptor (KIR3DL1) are found in 97% and 90% of individuals, respectively. These genes and their ligands are determined in the donor by KIR and HLA genotyping. We have generated a bank of EBV-accessory cells, which express all KIR ligands (C030) or totally lack their expression (721.221). Between these two opposite phenotypes, we have EBV-accessory cells that lack expression of only one MR ligand: HLA-C g1 (cell line PLH), HLA-C g2 (HOM-2), HLA-Bw4 (KT17), and HLA-A3/11 (Bri-P). The HLA-ligand mismatched EBV-accessory cell line may be thus use to induce preferential expansion of NK cells reactive to tumor cells lacking the selected HLA molecules. C030 and 721.221 cell lines may be used as negative and positive controls respectively

### EXAMPLE 2

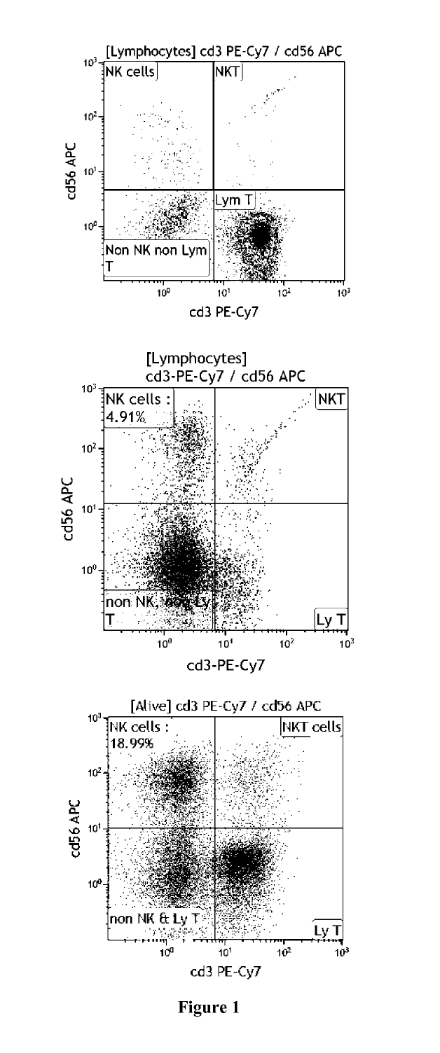

We hypothesized that by selecting the adequate EBV-cell line we can amplify a NK cell population expressing only a MR ligand. Results from two UCB units have confirmed this hypothesis. We obtained mononuclear cells from an mbilical cord blood (UCB) unit expressing HLA-Cg1 and HLA-Bw4 (see EXAMPLE 1). We incubate mononuclear cells with PLH, which lack HLA-Cg1 and KT17, which lack HLA-Bw4, cells in the presence of 100 U/ml of IL-2. We show that these accessory cells, i.e. KT17 or PLH, induce a large expansion of NK cells after ten days in co-culture (FIG. 1). Therefore, our accessory cells are immunologically competent to expand NK cells.

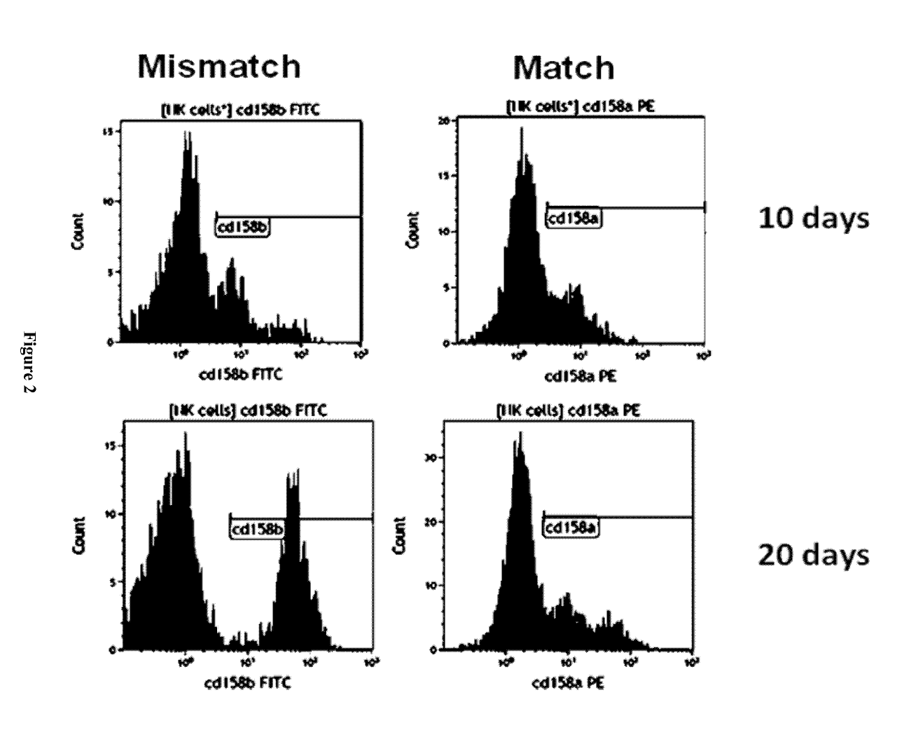

Moreover, PLH accessory cells, which lack HLA-Cg1, predominantly induce expansion of NK cells expressing KIR2DL2/3, which is the ligand of HLA-Cg1 alleles (FIG. 2). Ten days later we observed an increase of 17-times of expansion of NK cells expressing only KIR2DL2/3 when using PLH as accessory cells. Twenty days later this expansion reached 34 times. We did not observe this amplification when we used HOM-2 cells, which possessed HLA-C g1 ligands. However, HOM-2 cells increase 18-times NK cells expressing only KIR2DL1. Twenty days later this expansion reached 32 times. We did not observe this expansion in cells stimulated with PLH cells. Taken together these results show that it is possible to specifically direct NK cell expansion in vitro.

### EXAMPLE 3

Accessory or feeder cells induce a larger amplification of NK cells than interleukines. This is independent of the stimulation and the UCB. Other cell types such as T cells show minor expansion. NK cell expansion always works.

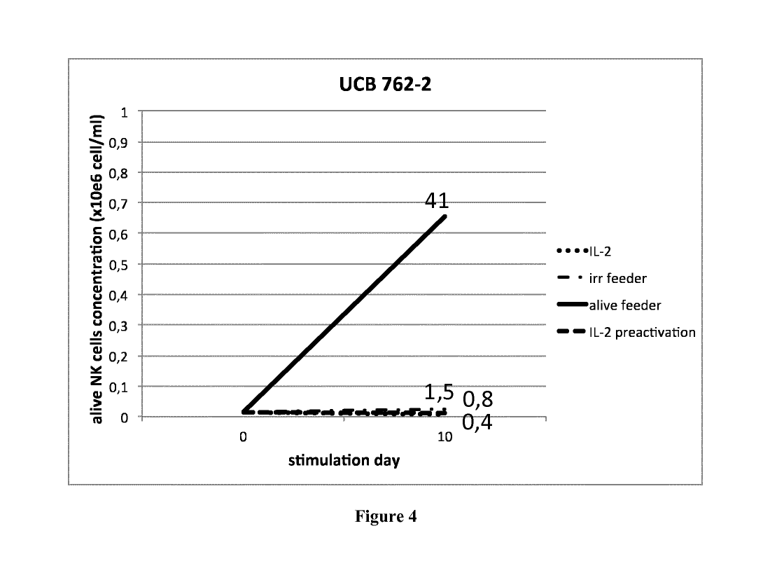

We obtain better results if we use the accesory/feeder cells alive (see FIG. 4). However it is necessary to optimize the protocol because NK cells need the continuous addition of feeder cells. In case of absence of re-stimulation with living cells, we observe better results with irradiated accessory cells. Our results suggest that depending the amount and frequency of alive feeder cells addition, the use of irradiated feeder cells or a small amount of alive feeder cells during the first 3 days greatly improve NK cell expansion.

Interestingly, our accessory cells keep NK cells expressing CD16. Therefore, these cells can mediate ADCC better than IL-2 expanded cells. In addition, accessory cells induce CD25 expression, whereas IL-2 fails to do it. The advantage is that adding IL-2 could keep CD25 expressing-cells in vivo in patients.

An important question is if our amplified NK cells show functional activity. The most relevant is cytolytic activity, which is almost absent in naïve NK cells. When growing with accessory cells, NK cells show CD107 degranulation. Importantly, when incubated overnight with their commonly used targets, they are still capable of increase CD107 degranulation up to 80%. This is similar or even higher that the classical result obtained with IL-2 stimulation that induces up to 70% of degranulation. In summary, accessory cells-amplified NK cells show a high degree of cytolytic activity.

### REFERENCES

Throughout this application, various references describe the state of the art to which this invention pertains. The disclosures of these references are hereby incorporated by reference into the present disclosure.

1. A method for preparing an accessory cell comprising the steps of i) providing a cell and ii) inhibiting in said cell the expression of one gene encoding for a Killer-Cell Immunoglobulin-like Receptor(s) (KIR) ligand.

2. The method according to claim. 1 which further comprises a step of inhibiting or reducing the MHC-I expression in said accessory cell.

3. The method according to claim 1 which further comprises a step of inhibiting the expression of ERK5 gene.

4. The method according to claim 1 which further comprises a step of immortalizing said accessory cell.

5. An accessory cell obtained by the steps of i) providing a cell and ii) inhibiting in said cell the expression of one gene encoding for a Killer-Cell Immunoglobulin-like Receptor(s) (KIR) ligand.

6. An in vitro or ex vivo method for preparing activated natural killer (NK) cells, comprising the steps
(i) contacting living NK cells with at least one accessory cell obtained by the steps of a) providing a cell and b) inhibiting in said cell the expression of one gene encoding for a Killer-Cell Immunoglobulin-like Receptor(s) (KIR) ligand, wherein said step of contacting is performed under conditions and for a duration sufficient to induce activation of the NK cells; and
(ii) recovering said activated NK cells.

7. A population of activated NK cells obtained by
(i) contacting living NK cells with at least one accessory cell obtained by the steps of a) providing a cell and b) inhibiting in said cell the expression of one gene encoding for a Killer-Cell Immunoglobulin-like Receptor(s) (KIR) ligand, wherein said step of contacting is performed under conditions and for a duration sufficient to induce activation of the NK cells; and
(ii) recovering said activated NK cells.

8. (canceled)

9. A method of treating cancer, infectious diseases or dysimmune diseases in a subject in need thereof, comprising
providing at least one accessory cell by inhibiting in at least one cell the expression of one gene encoding for a Killer-Cell Immunoglobulin-like Receptor(s) (KIR) ligand;
contacting living natural killer (NK) cells with said at least one accessory cell under conditions and for a duration sufficient to induce activation of the NK cells;
recovering activated NK cells; and.
administering said NK cells to said subject.
